SEQUENCE LISTING (5 sequences)

SEQ ID NO: 1 - DNA, 2823 bp, Artificial Sequence
  cDNA clone of MQ1 cell surface glycoprotein of Astrocytoma cell
gctcagaataccaatgactgcagccctcatccctgttacaacagcggcacctgtgtggatggagacaactggtaccggtgcgaatgtgccccgggttttgctgggcccgactgcagaataaacatcaatgaatgccagtcttcaccttgtgcctttggagcgacctgtgtggatgagatcaatggctaccggtgtgtctgccctccagggcacagtggtgccaagtgccaggaagtttcagggagaccttgcatcaccatggggagtgtgataccagatggggccaaatgggatgatgactgtaatacctgccagtgcctgaatggacggatcgcctgctcaaaggtctggtgtggccctcgaccttgcctgctccacaaagggcacagcgagtgccccagcgggcagagctgcatccccatcctggacgaccagtgcttcgtccacccctgcactggtgtgggcgagtgtcggtcttccagtctccagccggtgaagacaaagtgcacctctgactcctattaccaggataactgtgcgaacatcacatttacctttaacaaggagatgatgtcaccaggtcttactacggagcacatttgcagtgaattgaggaatttgaatattttgaagaatgtttccgctgaatattcaatctacatcgcttgcgagccttccccttcagcgaacaatgaaatacatgtggccatttctgctgaagatatacgggatgatgggaacccgatcaaggaaatcactgacaaaataatcgatcttgttagtaaacgtgatggaaacagctcgctgattgctgccgttgcagaagtaagagttcagaggcggcctctgaagaacagaacagatttccttgttcccttgctgagctctgtcttaactgtggcttggatctgttgcttggtgacggccttctactggtgcctgcggaagcggcggaagccgggcagccacacacactcagcctctgaggacaacaccaccaacaacgtgcgggagcagctgaaccagatcaaaaaccccattgagaaacatggggccaacacggtccccatcaaggattatgagaacaagaactccaaaatgtctaaaataaggacacacaattctgaagtagaagaggacgacatggacaaacaccagcagaaagcccggtttgccaagcagccggcgtacacgctggtagacagagaagagaagccccccaacggcacgccgacaaaacacccaaactggacaaacaaacaggacaacagagacttggaaagtgcccagagcttaaaccgaatggagtacatcgtatagcagaccgcgggcactgccgccgctaggtagagtctgagggcttgtagttctttaaactgtcgtgtcatactcgagtctgaggccgttgctgacttagaatccctgtgttaatttaagttttgacaagctggcttacactggcaatggtagtttctgtggttggctgggaaatcgagtgccgcatctcacagctatgcaaaaagctagtcaacagtaccctggttgtgtgtccccttgcagccgacacggtctcggatcaggctcccaggagcctgcccagccccctggtctttgagctcccacttctgccagatgtcctaatggtgatgcagtcttagatcatagttttatttatatttattgactcttgagttgtttttgtatattggttttatgatgacgtacaagtagttctgtatttgaaagtgcctttgcagctcagaaccacagcaacgatcacaaatgactttattatttattttttttaattgtatttttgttgttgggggaggggagactttgatgtcagcagttgctggtaaaatgaagaatttaaagaaaaaaatgtcaaaagtagaactttgtatagttatgtaaataattcttttttattaatcactgtgtatatttgatttattaacttaataatcaagagccttaaaacatcattcctttttatttatatgtatgtgtttagaattgaaggtttttgatagcattgtaagcgtatggctttatttttttgaactcttctcattacttgttgcctataagccaaaattaaggtgtttgaaaatagtttattttaaaacaataggatgggcttctgtgcccagaatactgatggaatttttttgtacgacgtcagatgtttaaaacaccttctatagcatcacttaaaacacgttttaaggactgactgaggcagtttgaggattagtttagaacaggtttttttgtttgtttgttttttgtttttctgctttagacttgaaaagagacaggcaggtgatctgctgcagagcagtaagggaacaagttgagctatgacttaacatagccaaaatgtgagtggttgaatatgattaaaaatatcaaattaattgtgtgaacttggaagcacaccaatcttactttgtaaattctgatttcttttcaccattcgtacataatactgaaccacttgtagatttgattttttttttaatctactgcatttagggagtattctaataagctagttgaatacttgaaccataaaatgtccagtaagatcactgtttagatttgccatagagtacactgcctgccttaagtgaggaaatcaaagtgctattacgaagttcaagatcaaaaaggcttataaaacagagtaatcttgttggttcaccattgagaccgtgaagatactttgtattgtcctattagtgttatatgaacatacaaatgcatctttgatgtgttgttcttggcaataaattttgaaaagtaatatttattaaa

SEQ ID NO: 2 - DNA, 2476 bp, Artificial Sequence
  cDNA clone of MQ-1 cell surface glycoprotein of Astrocytoma cell
aggtctggtgtggccctcgaccttgcctgctccacaaagggcacagcgagtgccccagcgggcagagctgcatccccatcctggacgaccagtgcttcgtccacccctgcactggtgtgggcgagtgtcggtcttccagtctccagccggtgaagacaaagtgcacctctgactcctattaccaggataactgtgcgaacatcacatttacctttaacaaggagatgatgtcaccaggtcttactacggagcacatttgcagtgaattgaggaatttgaatattttgaagaatgtttccgctgaatattcaatctacatcgcttgcgagccttccccttcagcgaacaatgaaatacatgtggccatttctgctgaagatatacgggatgatgggaacccgatcaaggaaatcactgacaaaataatcgatcttgttagtaaacgtgatggaaacagctcgctgattgctgccgttgcagaagtaagagttcagaggcggcctctgaagaacagaacagatttccttgttcccttgctgagctctgtcttaactgtggcttggatctgttgcttggtgacggccttctactggtgcctgcggaagcggcggaagccgggcagccacacacactcagcctctgaggacaacaccaccaacaacgtgcgggagcagctgaaccagatcaaaaaccccattgagaaacatggggccaacacggtccccatcaaggattatgagaacaagaactccaaaatgtctaaaataaggacacacaattctgaagtagaagaggacgacatggacaaacaccagcagaaagcccggtttgccaagcagccggcgtacacgctggtagacagagaagagaagccccccaacggcacgccgacaaaacacccaaactggacaaacaaacaggacaacagagacttggaaagtgcccagagcttaaaccgaatggagtacatcgtatagcagaccgcgggcactgccgccgctaggtagagtctgagggcttgtagttctttaaactgtcgtgtcatactcgagtctgaggccgttgctgacttagaatccctgtgttaatttaagttttgacaagctggcttacactggcaatggtagtttctgtggttggctgggaaatcgagtgccgcatctcacagctatgcaaaaagctagtcaacagtaccctggttgtgtgtccccttgcagccgacacggtctcggatcaggctcccaggagcctgcccagccccctggtctttgagctcccacttctgccagatgtcctaatggtgatgcagtcttagatcatagttttatttatatttattgactcttgagttgtttttgtatattggttttatgatgacgtacaagtagttctgtatttgaaagtgcctttgcagctcagaaccacagcaacgatcacaaatgactttattatttattttttttaattgtatttttgttgttgggggaggggagactttgatgtcagcagttgctggtaaaatgaagaatttaaagaaaaaaatgtcaaaagtagaactttgtatagttatgtaaataattcttttttattaatcactgtgtatatttgatttattaacttaataatcaagagccttaaaacatcattcctttttatttatatgtatgtgtttagaattgaaggtttttgatagcattgtaagcgtatggctttatttttttgaactcttctcattacttgttgcctataagccaaaattaaggtgtttgaaaatagtttattttaaaacaataggatgggcttctgtgcccagaatactgatggaatttttttgtacgacgtcagatgtttaaaacaccttctatagcatcacttaaaacacgttttaaggactgactgaggcagtttgaggattagtttagaacaggtttttttgtttgtttgttttttgtttttctgctttagacttgaaaagagacaggcaggtgatctgctgcagagcagtaagggaacaagttgagctatgacttaacatagccaaaatgtgagtggttgaatatgattaaaaatatcaaattaattgtgtgaacttggaagcacaccaatcttactttgtaaattctgatttcttttcaccattcgtacataatactgaaccacttgtagatttgattttttttttaatctactgcatttagggagtattctaataagctagttgaatacttgaaccataaaatgtccagtaagatcactgtttagatttgccatagagtacactgcctgccttaagtgaggaaatcaaagtgctattacgaagttcaagatcaaaaaggcttataaaacagagtaatcttgttggttcaccattgagaccgtgaagatactttgtattgtcctattagtgttatatgaacatacaaatgcatctttgatgtgttgttcttggcaataaattttgaaaagtaatatttat

SEQ ID NO: 3 - DNA, 2721 bp, Artificial Sequence
  Splice Variant
atgcgttccccacggacgcgcggccggtccgggcgccccctaagcctcctgctcgccctgctctgtgccctgcgagccaaggtgtgtggggcctcgggtcagttcgagttggagatcctgtccatgcagaacgtgaacggggagctgcagaacgggaactgctgcggcggcgcccggaacccgggagaccgcaagtgcacccgcgacgagtgtgacacatacttcaaagtgtgcctcaaggagtatcagtcccgcgtcacggccggggggccctgcagcttcggctcagggtccacgcctgtcatcgggggcaacaccttcaacctcaaggccagccgcggcaacgaccgcaaccgcatcgtgctgcctttcagtttcgcctggccgaggtcctatacgttgcttgtggaggcgtgggattccagtaatgacaccgtcgaacatcacatttacctttaacaaggagatgatgtcaccaggtcttactacggagcacatttgcagtgaattgaggaatttgaatattttgaagaatgtttccgctgaatattcaatctacatcgcttgcgagccttccccttcagcgaacaatgaaatacatgtggccatttctgctgaagatatacgggatgatgggaacccgatcaaggaaatcactgacaaaataatcgatcttgttagtaaacgtgatggaaacagctcgctgattgctgccgttgcagaagtaagagttcagaggcggcctctgaagaacagaacagatttccttgttcccttgctgagctctgtcttaactgtggcttggatctgttgcttggtgacggccttctactggtgcctgcggaagcggcggaagccgggcagccacacacactcagcctctgaggacaacaccaccaacaacgtgcgggagcagctgaaccagatcaaaaaccccattgagaaacatggggccaacacggtccccatcaaggattatgagaacaagaactccaaaatgtctaaaataaggacacacaattctgaagtagaagaggacgacatggacaaacaccagcagaaagcccggtttgccaagcagccggcgtacacgctggtagacagagaagagaagccccccaacggcacgccgacaaaacacccaaactggacaaacaaacaggacaacagagacttggaaagtgcccagagcttaaaccgaatggagtacatcgtatagcagaccgcgggcactgccgccgctaggtagagtctgagggcttgtagttctttaaactgtcgtgtcatactcgagtctgaggccgttgctgacttagaatccctgtgttaatttaagttttgacaagctggcttacactggcaatggtagtttctgtggttggctgggaaatcgagtgccgcatctcacagctatgcaaaaagctagtcaacagtaccctggttgtgtgtccccttgcagccgacacggtctcggatcaggctcccaggagcctgcccagccccctggtctttgagctcccacttctgccagatgtcctaatggtgatgcagtcttagatcatagttttatttatatttattgactcttgagttgtttttgtatattggttttatgatgacgtacaagtagttctgtatttgaaagtgcctttgcagctcagaaccacagcaacgatcacaaatgactttattatttattttttttaattgtatttttgttgttgggggaggggagactttgatgtcagcagttgctggtaaaatgaagaatttaaagaaaaaaatgtcaaaagtagaactttgtatagttatgtaaataattcttttttattaatcactgtgtatatttgatttattaacttaataatcaagagccttaaaacatcattcctttttatttatatgtatgtgtttagaattgaaggtttttgatagcattgtaagcgtatggctttatttttttgaactcttctcattacttgttgcctataagccaaaattaaggtgtttgaaaatagtttattttaaaacaataggatgggcttctgtgcccagaatactgatggaatttttttgtacgacgtcagatgtttaaaacaccttctatagcatcacttaaaacacgttttaaggactgactgaggcagtttgaggattagtttagaacaggtttttttgtttgtttgttttttgtttttctgctttagacttgaaaagagacaggcaggtgatctgctgcagagcagtaagggaacaagttgagctatgacttaacatagccaaaatgtgagtggttgaatatgattaaaaatatcaaattaattgtgtgaacttggaagcacaccaatcttactttgtaaattctgatttcttttcaccattcgtacataatactgaaccacttgtagatttgattttttttttaatctactgcatttagggagtattctaataagctagttgaatacttgaaccataaaatgtccagtaagatcactgtttagatttgccatagagtacactgcctgccttaagtgaggaaatcaaagtgctattacgaagttcaagatcaaaaaggcttataaaacagagtaatcttgttggttcaccattgagaccgtgaagatactttgtattgtcctattagtgttatatgaacatacaaatgcatctttgatgtgttgttcttggcaataaattttgaaaagtaatatttattaa

SEQ ID NO: 4 - DNA, 18 bp, Artificial Sequence
  Antisense Oligonucleotide
tggggaacgcatcgctgc

SEQ ID NO: 5 - DNA, 18 bp, Artificial Sequence
  Antisense Control Oligonucleotide
tggggaccgcatcgctgc

(19) United States
(12) Patent Application Publication
(10) Pub. No.: US 2007/0105164 A1
(43) Pub. Date: May 10, 2007

(54) Cancer diagnosis and therapy

(75) Inventors: Karl Mulligan, Belfast (GB); Derek McCormick, Belfast (GB); Patrick Johnston, Belfast (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: The Queen's University of Belfast, Balfast (GB)

(21) Appl. No.: 10/579,168

(22) PCT Filed: Nov. 15, 2004
(86) PCT No.: PCT/GB04/04788

§ 371 Date: Aug. 18, 2006

(30) Foreign Application Priority Data
Nov. 14, 2003 (GB) 0326578.2

(51) Int. Cl. G01N 33/574; C07H 21/04; C12P 21/06; C07K 16/30; C07K 14/82

(52) U.S. Cl. 435/7.23; 435/69.1; 435/320.1; 435/325; 530/388.8; 530/395; 536/23.5

(57) ABSTRACT

The invention relates to an oncofetal glycoprotein, referred to as MQ-1, nucleic acid sequences coding for the protein, and antibodies which bind specifically to the protein. Also described is a hybridoma capable of producing monoclonal antibodies which bind specifically to the protein of the invention. Methods, and kits, for diagnosing and treating cancer using the antibodies of the invention are also described. Anti-sense polynucleotides are also described, as are methods for inducing apoptosis in cells which express MQ-1.

### TECHNICAL FIELD

The invention relates to a novel oncofetal glycoprotein which is expressed in certain tumours, antibodies to the protein, and uses of the antibodies in cancer diagnosis.

### BACKGROUND ART

The cancer phenotype typically displays loss of differentiation, loss of proliferative control and altered cell adhesion molecule expression. Cell surface proteins have been shown to play an important role in cell-cell interactions (eg NCAM), cell-extra-cellular interactions (eg CD44) and cell regulation (eg Notch signaling).

Some of these cell surface proteins have oncofetal expression profiles and as such have been used as tumour specific diagnostic markers (eg CEA). A further use for antibodies specific for cell surface proteins over expressed in cancer has been in the treatment of cancer by immunotherapy/radioimmunotherapy (eg Herceptin an antibody recognizing HER2).

Statements of Invention

In one aspect, the invention relates to an isolated nucleic acid sequence which comprises a sequence selected from the group consisting of: Sequence ID No.1, Sequence ID No, 2, and Sequence ID No. 3. Typically, the nucleic acid sequence is a DNA sequence. In one embodiment, the isolated nucleic acid sequence consists of a sequence selected from the group consisting of: Sequence ID No. 1, Sequence ID No. 2 and Sequence ID No. 3.

The invention also relates to an isolated protein encoded by the isolated nucleic acid sequences of the invention, or a fragment or analogue therof. Typically, the protein is a cell surface glycoprotein. In one preferred embodiment, the isolated protein is an oncofetal protein expressed by an astrocytoma cell. Typically, the protein has a molecular weight of approximately 200 kda. In this specification, the term “protein” should be understood as including amino acid sequences which would more generally be referred to a peptides.

In another aspect, the invention relates to an antibody which binds specifically to the protein of the invention and any other antibody that competes directly or by stearic hindrance therewith for said protein. Typically, the antibody is a monoclonal antibody. In one embodiment, the antibody is a class M immunoglobulin with a kappa-light chain.

In another aspect, the invention relates to a fragment of the antibody of the invention, which fragment binds specifically to the protein of the invention.

In another aspect, the invention relates to a method of producing an antibody to a protein comprising:
innoculating an animal with a protein according to the invention, wherein the protein elicits an immune response in the animal to produce the antibody; and
isolating the antibody from the animal.

In one embodiment, the animal is innoculated with G-CCM cells of ECACC deposit No. 86022702.

In a further aspect, the invention relates to a process for producing a hybridoma, comprising the step of innoculating a suitable subject with a protein according to the invention, or an antigenic fragment thereof, and fusing cells from the subject with a myeloma cell to produce the hybridoma. Typically, the subject is innoculated with G-CCM cells of ECACC deposit No. 86022702.

In a further aspect, the invention relates to a hybridoma cell obtainable according to the above process. In one embodiment, the invention relates to a hybridoma cell of, or derived from, ECACC Deposit No. 03073001.

A deposit of hybridoma cells according to the invention was made at the European Collection of Cell Cultures on 30 Jul. 2003 and accorded the accession number ECACC 03073001.

In another aspect, the invention relates to a monoclonal antibody obtainable from a hybridoma cell of, or derived from, ECACC Deposit No. 03073001.

The invention also relates to a method of detecting an astrocytoma cell in a sample of human cells, which method comprises the step of contacting the cell sample with an antibody of the invention, or a fragment thereof, and detecting those cells which have bound the antibody or fragment, wherein binding of the antibody or the fragment to a cell is indicative of an astrocytoma cell. Typically, the antibody is a monoclonal antibody of the invention.

The invention also relates to a method of detecting a primary breast carcinoma cell in a sample of human cells, which method comprises the step of contacting the cell sample with an antibody of the invention, or a fragment thereof, and detecting those cells which have bound the antibody or fragment, wherein binding of the antibody or the fragment to a cell is indicative of a primary breast carcinoma cell. Typically, the antibody is a monoclonal antibody of the invention.

The invention also relates to a diagnostic kit for diagnosing the presence of a cell selected from the group consisting of: astrocytoma cells; malignant melanoma secondary tumour cells; and primary breast carcinoma cells, the kit comprising an antibody according to the invention, or a fragment thereof. Typically, the antibody is a monoclonal antibody of the invention. In one embodiment, the antibody of the invention comprises a detectable label. Alternatively, the kit comprises a secondary antibody which specifically binds the antibody of the invention, which secondary antibody comprises a detectable label.

The invention also relates to a biological targeting device comprising an antibody, typically a monoclonal antibody, of the invention, or a fragment thereof, and a therapuetic ligand.

The invention also relates to a therapeutic antibody comprising an antibody, typically a monoclonal antibody, of the invention, or a fragment thereof.

The invention also relates to a method of treating cancer in an individual by inducing apoptosis in cells in the individual which express a protein of the invention, which method comprises a step of treating an individual with an antibody of the invention, or a fragment thereof. Typically, the antibody is a monoclonal antibody. In one embodiment, the cancer is selected from the group consisting of: malignant astrocytomas; malignant melanoma secondary tumours; and primary breast carcinomas. Typically, the antibody is humanised.

The invention also relates to a polynucleotide which is anti-sense to at least a portion of an insolated nucleic acid sequence of the invention. Typically, the polynucleotide is anti-sense to all or part of a transcription initiator site of the isolated nucleic acid sequence of the invention. In one embodiment, the anti-sense polynucleotide comprises, or consists of, a sequence of Sequence ID No. 4.

The invention also relates to a method of treating cancer in an individual by inducing apoptosis in cells in the individual which express a protein of the invention, which method comprises a step of treating an individual with an anti-sense polynucleotide of the invention. In one embodiment, the cancer is selected from the group consisting of: malignant astrocytomas; malignant melanoma secondary tumours; and primary breast carcinomas. Methods of delivery of anti-sense polynucleotides will be well-known to those skilled in the art of gene therapy.

The monoclonal antibodies of the invention may be the complete antibodies described herein, or fragments thereof. That is, they may be any fragment of a monoclonal antibody of the invention that specifically recognises the protein of the invention. Such fragments include Fab, F(ab′)2, Fab′, etc. These fragments ban be prepared by digestion with an enzyme such as papain, pepson, ficin, or the like. The properties of the obtained fragments can be confirmed in the same manner as described herein.

The principle reason for the poor prognosis associated with malignant astrocytomas is recurrence due to invasion of surrounding brain parenchyma by tumour cells with an invasive phenotype. This phenotype displays loss of differentiation, secretion of proteases and altered cell adhesion molecule expression. As part of an investigation into the mechanisms of astrocytoma invasion, monoclonal antibodies (Mab) were raised against cell surface proteins expressed by an anaplastic astrocytoma cell line (G-CCM). One of the antibodies produced (MQ1 Mab) recognizes a previously undescribed cell surface glycoprotein (MQ1). In vitro MQ1 protein expression was found on astrocytomas and fetal astrocytes, with the level of expression increasing with astrocytoma malignancy and decreasing with fetal astrocyte maturity. Immunohistochemistry on histologically normal and neoplastic brain tissue demonstrated that MQ1 protein expression is restricted to astrocytomas (n=52). Other primary brain tumours tested (oligodendrogliomas, neurinomas, PNET, and medulloblastomas) and normal brain cells, including neurons, oligodendrocytes and endothelial cells were MQ1 negative, thus indicating that the MQ1 proteins have the expression pattern of oncofetal proteins. Similarily a study looking at primary breast carcinomas found 60% were MQ1 positive (n=228). Surrounding normal tissue, fibrocystic disease and fibroadenoma tissue were MQ1 negative. Malignant melanoma secondary tumours to the brain were also found to be strongly MQ1 positive.

A CDNA expression library was synthesized from G-CCM mRNA and screened with the MQ1 antibody. Two positive clones were isolated (Sequence ID No.s 1 and 2) and sequencing data demonstrated that both have a high degree of homology with Jagged1, a human Notch ligand which plays a role in differentiation and determination of cell fate. The library was rescreened with probes generated from the positive clones and further homologous transcripts were isolated including a possible Jagged1 splice variant (Sequence ID No. 3). Northern blotting for a range of cell lines with these probes revealed the presence of two transcripts (approximately 3.5 kb & 5.0 kb). Subsequent protein studies (immunocytochemistry, immunoblotting and co-immunoprecipitation) indicate that the MQ1 protein has a high degree of homology with, but is not identical to, Jagged1.

This investigation has identified a novel oncofetal glycoprotein with homology to Jagged1. Its tumour specificity together with its potential role in regulating cellular differentiation/apoptosis suggest that it may be a valuable prognostic marker and therapeutic target.

The invention will be more clearly understood from the following description of some embodiments thereof, given by way of example only, with reference to the following Figures in which:

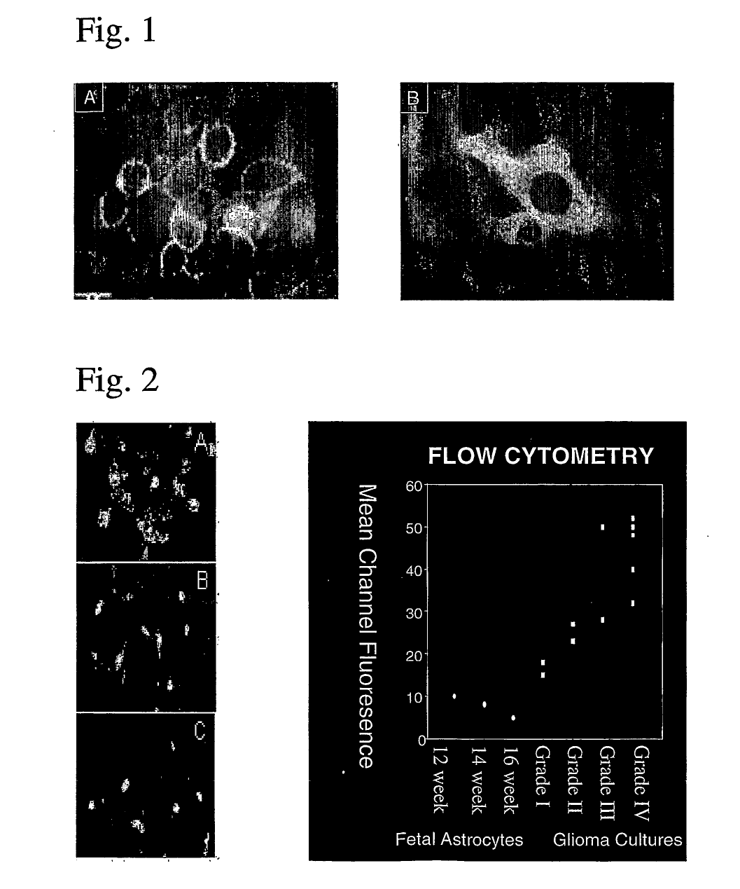

FIG. 1A illustrates confocal microscopy of live G-CCM cells immunolabelled with MQ1 showing recognition of a cell surface epitope;

FIG. 1B illustrates confocal microscopy of permeabilized G-CCM cells immunolabelled with MQ1 showing recognition of an intracellular epitope and localisation of the antigen at areas of cell contact on the cell surface;

FIG. 2 shows a comparision of MQ1 expression, by immunocytochemistry and flow cytometry, on a range of fetal astrocyte cultures and astrocytoma cell lines. A-C show immunocytochemistry on live cells of a grade IV, grade III and 16 week gestation fetal astrocytes respectively. D-F chow the corresponding flow analysis with the same cells with the level of MQ1 surface expression estimated as mean channel fluorescence. G shows the results of the flow analysis plotted as a graph. This demonstrates an inverse correlation of cell surface MQ1 protein expression with fetal astrocyte maturity and correlation with astrocytoma grade;

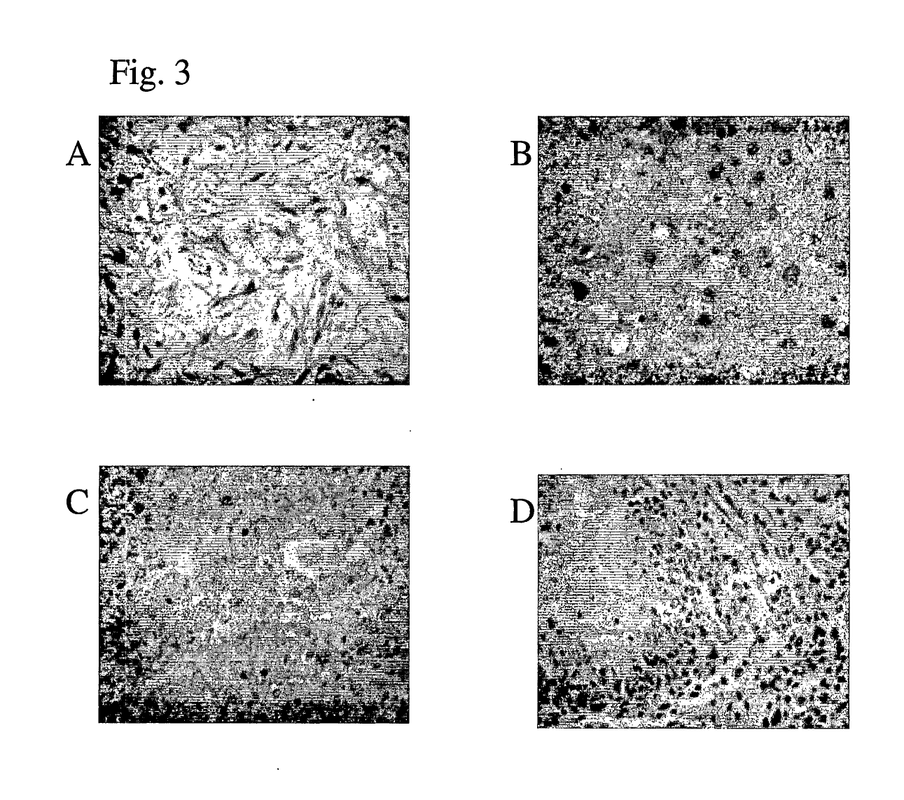

FIG. 3 shows immunohistochemistry displaying diffuse MQ1 positivity throughout A) Grade I astrocytomas B) Grade II astrocytomas C) Grade III astrocytomas & D) focal positivity in grade IV astrocytoma cells palisading an area of necrosis;

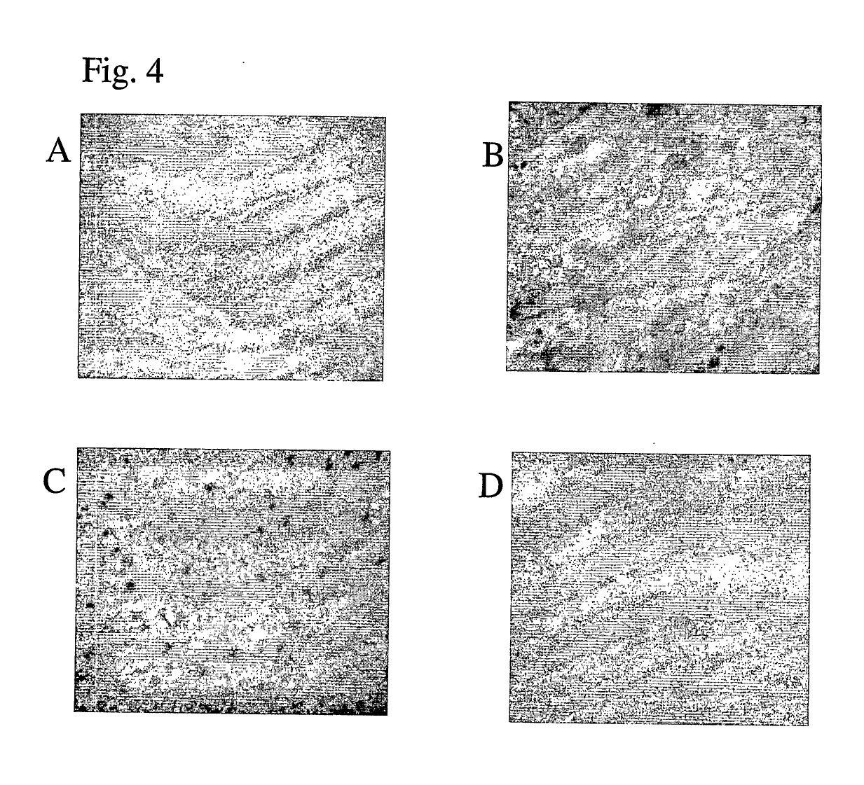

FIG. 4 shows MQ1 immunocytochemistry showing A) strong MQ1 positivity at the tumour front B) strong MQ1 positivity in reactive astrocytes in adjacent tissue C) GFAP positivity in reactive MS tissue D) MQ1 negative reactive MS tissue;

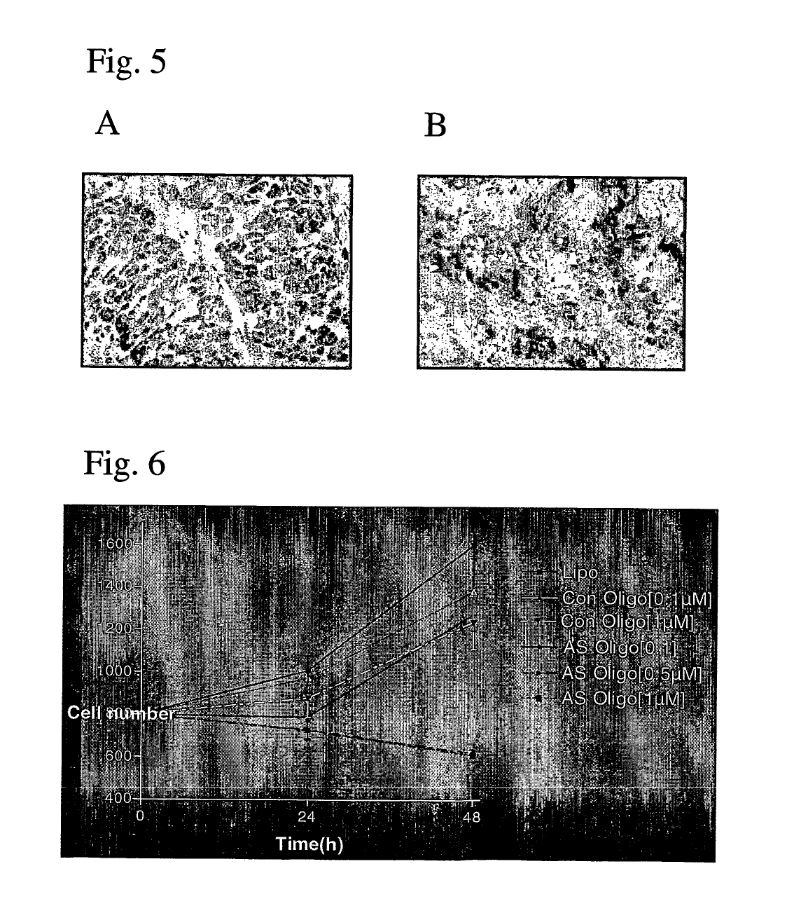

FIG. 5 shows MQ1 immunohistochemistry of breast carcinoma tissue showing A) strong MQ1 positivity in invasive ductal carcinoma cells surrounded by MQ1 negative stroma B) strong MQ1 positivity in lobular carcinoma surrounded by MQ1 negative stroma;

FIG. 6 shows MQ1 immunocytochemistry of G-CCM cells treated with (A) 0.1 μm control oligo (B) 0.5 μm control oligo (C) 1.0 μm control oligo (D) 0.1 μm anti-sense MQ1 oligo (E)0.5 μm anti-sense MQ1 oligo and (F) 1.0 μm antisense oligo, showing that MQ1 anti-sense oligo knocks out MQ1 protein expression at concentrations of 0.5 and 1.0 μm;

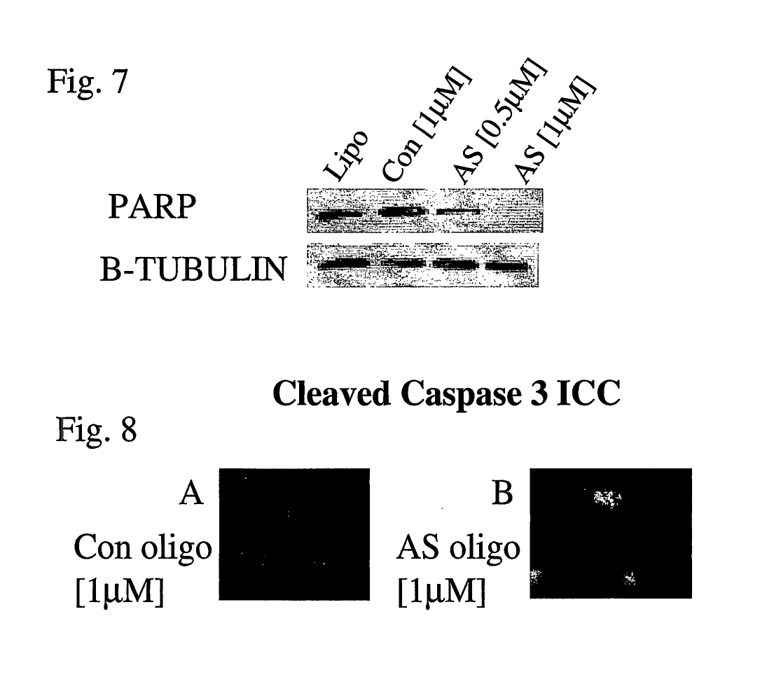

FIG. 7 shows an immunoblot indicating Parp cleavage of oligo-treated G-CCM cells;

FIG. 8 shows immunocytochemistry (ICC) detection of cleaved Caspase 3 following oligo treatmentl; and

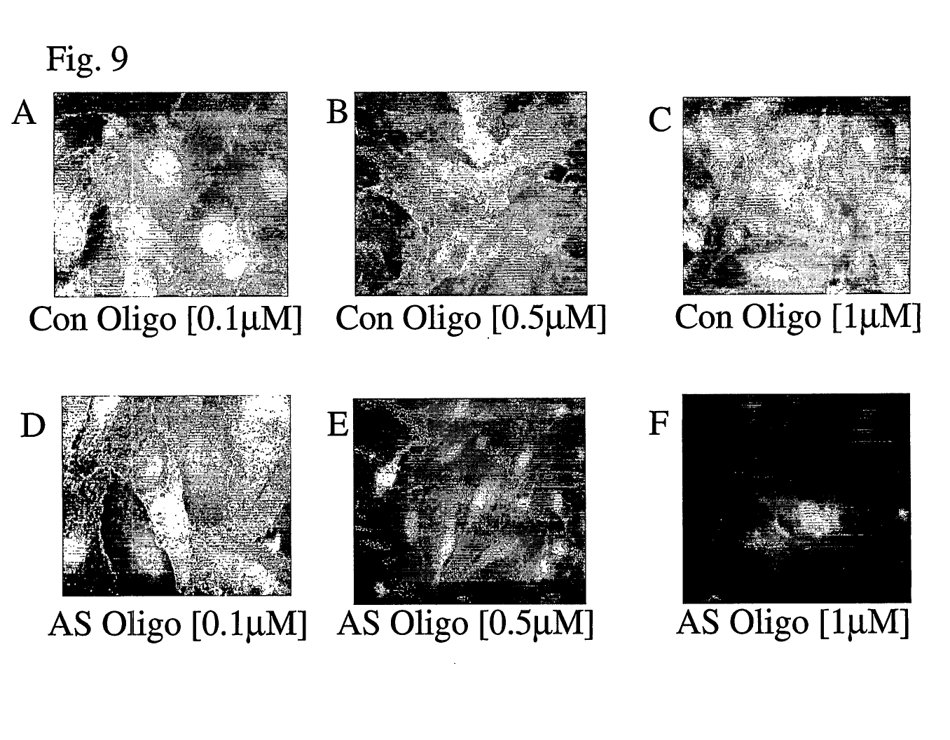

FIG. 9 shows G-CCM cells labelled with MQ1 antibody by ICC, 24 hours post-treatment with control and anti-sense oligonucleotides in which:
(A) control oligo 0.1 μM
(B) control oligo 0.5 μM
(C) control oligo 1.0 μM
(D) Anti-sense oligo 0.1 μM
(E) Anti-sense oligo 0.5 μM
(F) Anti-sense oligo 0.1 μM

### MATERIALS AND METHODS

Materials

All cell culture reagents were obtained from Gibco BRL (Paisley, UK) with the exception of the hypoxanthine, aminopterin and thymidine (HAT) and the hypoxanthine and thymidine (HT) that were obtained from Sigma (Poole, Dorset, UK). The secondary and negative control antibodies were supplied by Dako (Bucks, UK). The PARP and Caspase3 antibodies were purchased from Sigma (Poole, Dorset, UK) and the Protein-A Sepharose CL4B from Pharmacia Biotech (Herts, UK). PTO linked oligonucleotides were obtained from MWG-Biotech (Germany).

Cell culture

The CB109 cell line was established from a glioblastoma multiforme [6] and was a gift from Dr Claude Chauzy (Centre Henri Becquerel, Rouen, France). The G-CCM cell line was derived from a human anaplastic astrocytoma and was a gift from Dr Ian Freshney (Department of Clinical Oncology, University of Glasgow, UK). The G-CCM cell line is commercially available from the European Collection of Cell Cultures under Deposit No 86022702. The fetal astrocyte cell cultures were a gift from Ms Kim Martin (Department of Neuropathology, Institute of Psychiatry, London, UK). The C6 cell line, derived from a rat glioma, was obtained from Flow Laboratories (Scotland, UK). The skin fibroblast cell culture was initiated in our laboratory from a surgical specimen obtained from the Neurological Unit (Royal Victoria Hospital, Belfast, UK). The remaining glioma cell lines were initiated in our laboratory from surgical specimens received from the Neurosurgical Unit (Royal Victoria Hospital, Belfast, UK) and were used experimentally after 5-10 passages. Tumour grading follows the World Health Organisation classification. Cell lines were incubated at 37° C./5% CO2 in Dulbecco's modified Eagle's medium (DMEM) containing 2 mM glutamine, 10% fetal calf serum (FCS), and phenol red. All cell lines were tested for mycoplasma using Hoechst 33258 fluorescent dye and were found to be negative.

Monoclonal Antibody Production

Mabs were produced utilizing a standardized protocol designed to promote a rapid predominantly IgG response. In brief, a BALB/c mouse was inoculated intra-peritoneally with 5×106 G-CCM cells in 1 ml of Freund's complete adjuvant. Similar doses emulsified in Freund's incomplete adjuvant were administrated 14 and 28 days later to boost the immune response. Four days after the final booster inoculation the mouse was killed, its spleen aseptically removed and the splenocytes induced to fuse with NSO myeloma cells (at a ratio 5:1) using polyethylene glycol. The resulting fusion products were suspended in a selective, HAT-supplemented, growth medium (RPMI-1640 medium containing 10 mM L-glutamine, 1% sodium pyruvate, 100 iu/ml penicillin, 100 □g/ml streptomycin and 20% Myoclone FCS) and seeded into 96-well plates. The medium, from the viable hybridomas produced, was screened by indirect immunofluorescence against live and acetone-fixed G-CCM cells. Those showing specific recognition were recloned three times, to ensure monospecificity, in HT-supplemented growth medium and stored in liquid nitrogen. The hybridoma cell line MQ-1, which produced an antibody recognizing a cell surface antigen was propagated as an ascitic tumour in BALB/c mice previously immunosuppressed with Pristane. The ascitic fluids were collected, centrifuged and frozen at −20° C. until use.

The positively labelling Mabs were isotyped for their class and light chains using a monoclonal antibody isotyping kit.

Immunofluorescence

Hybridoma medium (neat) or ascites fluid (diluted 1:200 in PBS) was incubated with living cells, grown to 90% confluence on coverslips, for 40 min at room temperature (RT). After washing, the cells were fixed in acetone at −20° C. for 10 min followed by rehydration in PBS and incubation with an FITC-conjugated rabbit antimouse antibody (FITC-RAM) for 30 min at RT. After two further washes the cells were mounted on a glass slide, in a drop of Citifluor, and examined using a Zeiss immunofluorescence microscope or a Biorad confocal microscope. Incubations in PBS without primary antibody were used as negative controls. The fluorescent labelling of positive cells was subjectively rated from low intensity (+) to high intensity (++++).

Flow Cytometry

A preliminary study (results not shown) comparing the expression of MQ-1 protein on cells removed enzymatically (trypsin) and non-enzymatically (0.53 mM EDTA in PBS) from culture flasks, revealed that the MQ1 protein epitope was trypsin-resistant.

Cultured cells were removed from the flasks by trypsinization, counted and aliquoted into centrifuge tubes at a concentration of 5×105 cells per tube. Triplicate samples were incubated in excess ascitic fluid in 200 □l of serum free medium containing 1% bovine serum albumin (SFM/BSA) for 40 min at RT with gentle agitation. Following 2 washes in SFM the cells were incubated in an FITC-RAM antibody for 30 min at RT with gentle agitation. The cells were then washed twice in SFM and fixed in PBS containing 1% para-formaldehyde. The samples were analysed within 48 hr of fixation, using a Coulter EPICS Elite flow cytometer. Negative controls were incubated with an antibody raised against Aspergillus niger glucose oxidase, an enzyme not present or inducible in mammalian cells. The consistency of the mean channel fluorescence measurements between sample batches was checked using EPICS Immuno-Brite standards.

Immunohistochemistry

On receipt the tissue was fixed in 10% formalin prior to routine embedding in paraffin wax using a Tissue Tex VIP (Miles Scientific) automated processor. The paraffin blocks were sectioned at a thickness of 6 mm and mounted onto 3-aminopropyltriethoxysilane-coated slides. The tissue sections for indirect immunohistochemistry were processed using an avidin-biotin peroxidase complex (ABC) method. The tissue was dewaxed in xylene and rehydrated before endogenous peroxidase activity was blocked by a 10 min incubation in 3% H2O2 in methanol at room temperature (RT). To counter antigen masking, due to the formalin fixation, the tissue was pretreated with microwave irradiation to promote antigen retrieval. The sections were washed in distilled water and placed in 0.01M Tri-Na citrate pH7.8 and irradiated in a Miele microwave oven for 6 min (2×3 min) at 450 W (the optimal toime and intensity of irradiation was determined from preliminary studies). After incubation in PBS containing 5% normal rabbit serum for 10 min at RT the sections were incubated in MQ1 ascites (diluted 1:50 in PBS) at 4C overnight. Following 2×5 min washes in PBS the sections were incubated in biotinylated rabbit anti-mouse IgM diluted 1:400 in PBS for 40 min at RT. After further washes in PBS, a streptavidin-biotin complex linked to peroxidase was added to the sections and incubated for 40 min at RT. The peroxidase reaction was developed in 0.1% diaminobenzidine in PBS activated with 1% H2O2. After washing in water, the sections were counterstained in haematoxylin, dehydrated through graded alcohols, cleared in xylene and mounted in DPX. In addition to negative controls, incubated with a primary antibody raised against Aspergillus niger glucose oxidase, positive controls of histologically normal brain and astrocytoma tissue were included with every batch. cDNA Expression Library and screening.

G-CCM Cell cDNA Library Synthesis

A Total RNA Isolation from G CCM Cells

This was performed using Tel-Test RNA Stat-60, following their guidelines. Web Site www.isotexdiagnostics.com/rna stat-60 reagent.html

B mRNA Purification from Total RNA

This was performed using Invitrogen's FastTrack 2.0 Kit, following their guidelines. Web Site www.invitrogen.com/content.cfm?pageid=3443&cfid=3308 35&cftoken=53475959#FastTrack

C cDNA Library Synthesis from mRNA

This was performed using a Stratagene cDNA synthesis kit (following their protocol). Stratagene ZAP Express cDNA Synthesis Kit Instruction Manual www.stratagene.com/manuals/200403.pdf

Results

Antibody Production

The fusion resulted in the production of five viable antibody secreting hybridomas which screened positively by immunofluorescence microscopy on acetone fixed G-CCM cells. Of these, one (hybridoma MQ1) was found to secrete an antibody which was isotyped as a class M immunoglobulin with a kappa-light chain. This antibody recognizes a cell surface epitope, showing punctate labelling, on live G-CCM cells. Further examination by confocal microscopy confirmed the cell surface labelling of live G-CCM cells and revealed the presence of an intra-cellular epitope in permeabilized cells (FIGS. 1A&B). In addition examination of the permeabilized cells demonstrated localisation of labelling at focal adhesion points on the cell surface.

Immunocytochemistry

A range of cell lines was examined by indirect immunofluorescence for the presence of the MQ-1 antigen (Table 1).

TABLE 1

| CELL LINE | TISSUE SOURCE | MQ1 LABELLING |
|---|---|---|
| Fibroblasts | Normal skin | — |
| C6 | Rat glioma | — |
| FA 10 weeks | Human fetal astrocytes | + |
| FA 12 weeks | Human fetal astrocytes | + |
| FA 14 weeks | Human fetal astocytes | + |
| FA 15 weeks | Human fetal astrocytes | + |
| FA 16 weeks | Human fetal astrocytes | + |
| FA 19 weeks | Human fetal astrocytes | + |
| NP 527/94 | Pilocytic astrocytoma (I) | ++ |
| NP 396/94 | Pilocytic astrocytoma (I) | ++ |
| NP 424/94 | Astrocytoma (II) | ++ |
| NP 676/92 | Astrocytoma (II) | ++ |
| NP 445/92 | Astrocytoma (II) | ++ |
| NP 204/92 | Astrocytoma (II) | ++ |
| NP 482/96 | Astrocytoma (II) | ++ |
| NP 473/92 | Anaplastic astrocytoma (III) | +++ |
| G-CCM | Anaplastic astrocytoma (III) | ++++ |
| NP 493/94 | Anaplastic astrocytoma (III) | +++ |
| NP 785/96 | Anaplastic astrocytoma (III) | +++ |
| NP 402/93 | Glioblastoma multiforme (IV) | ++++ |
| NP 293/96 | Glioblastoma multiforme (IV) | +++ |
| NP 602/91 | Glioblastoma multiforme (IV) | ++++ |
| NP 536/94 | Glioblastoma multiforme (IV) | +++ |
| NP 306/92 | Glioblastoma multiforme (IV) | ++++ |
| NP 479/95 | Glioblastoma multiforme (IV) | +++ |
| NP 770/96 | Glioblastoma multiforme (IV) | +++ |
| NP 876/96 | Glioblastoma multiforme (IV) | ++++ |
| NP 39/96 | Glioblastoma multiforme (IV) | +++ |
| CB 109 | Glioblastoma multiforme (IV) | — |
| NP 670/92 | Glioblastoma multiforme (IV) | — |

Table 1. Indirect immunofluorescence on a range of live cell lines and cell cultures with MQ1 antibody.

The results show that the human skin fibroblasts and the C6, rat glioma, cell lines do not express the antigen. The fetal astrocytes and glioma cell lines were positive with the exception of two cell lines (CB109 and NP670/92) derived from glioblastomas multiforme. Under subjective microscopic analysis there appeared to be a variation in labelling intensity between the positive cell lines. The high grade gliomas had a higher labelling intensity than low grade gliomas and fetal astrocytes. This was confirmed by flow cytometry (FIG. 2). The results show a progressive increase in MQ-1 antigen expression, as estimated by the mean channel fluorescence, from low to high grade astrocytomas, the expression on grade IV astrocytomas being more than double that of grade I astrocytomas. The fetal astrocytes showed a lower expression than the astrocytoma cell lines, that halved from fetal astrocytes of 12 weeks gestation to 16 weeks gestation.

Immunohistochemistry

The results of the immunohistochemical study on primary brain tumours are summarized in Table 2.

TABLE 2

| Tumour | # Biopsies | MQ1 positivity |
|---|---|---|
| Astrocytomas | 30 | 29/30 |
| Neurinoma | 3 | 0/3 |
| Oligodendroglioma | 3 | 0/3 |
| Medulloblastoma | 3 | 0/3 |
| PNET | 3 | 0/3 |

Table 2 Immunohistochemical analysis of MQ1 immuno-labelling of a range of Primary Brain Tumours showing that of the tumour tissue tested only astrocytomas displayed MQ1 positivity.

The results show that of all the primary brain tumours tested (oligodenrogliomas, PNET etc) only astrocytomas were MQ1 positive. All pilocytic (grade I) astrocytomas showed a similar staining pattern. There was strong cellular immunostaing of MQ1 proteins which extended to the cellular processes of bipolar cells (FIG. 3A). The immunopositive cells stood out prominently against a loosely arranged less cellular stroma. The astrocytomas (grade II) and anaplastic (grade III) astrocytomas revealed a diffuse immunopositivity and the staining pattern was similar in all (FIGS. 3B&C). There was variation in the staining pattern of glioblastomas. Out of 16 glioblastomas tested, 1 was unreactive revealing no MQ1 protein expression whereas 14 showed focal positivity and one diffuse immunostaining (FIG. 3D). Focal positivity was observed as clusters or groups of positive cells surrounded by unreactive aresas. Tumour cells palisading around areas of necrosis, a characteristic feature of glioblastomas also reveled focal positivity. However tumour giant cells, bizarre cells and clusters of proliferating endothelial cells were negative for MQ1 protein expression. The oligodendroglial cells were negative. Within adjacent grey matter the neurones did not show immunolabelling for the MQ1 proteins. The endothelial cells lining small and large blood vessels in and around tumours of all grades showed no MQ1 protein expression. There was no immunolabelling of lymphocytes in the perivascular spaces. The infiltrating edge of the tumours and the adjacent glial areas showed prominent labelling of large reactive astrocytes (FIGS. 4A&B)). Such cells revealed multiple processes. However this MQ1 positivity in reactive astrocytes was only found surrounding MQ1 positive tumours, other reactive tissue such as MS tissue that shows prominent reactive astrocytes when labeled for GFAP (FIG. 4C) displayed no MQ1 positivity in the 10 biopsies tested (FIG. 4D).

In non-CNS tissue tested malignant melanoma and breast 20 to the brain were found to express the MQ1 proteins (Table 3).

TABLE 3

| Tissue | # Biopsies | MQ1 Positivity |
|---|---|---|
| Breast 20 (brain) | 3 | 3/3 |
| Breast 1o | 228 | 137/228 |
| Fibroadenoma | 5 | 0/5 |
| Fibrocystic Diease | 5 | 0/5 |
| M. Melanoma2o (brain) | 4 | 4/4 |

Table 3 Immunohistochemical MQ1 immunolabelling of a range of non-CNS tumours, showing MQ1 positivity in 60% of primary breast tumours and no positivity in fibrocystic diease and fibroadenomas that are non-malignant breast conditions.

Of the primary breast tumours tested 137/228 were MQ1 positive while fibrocystic diease and fibroadenoma tissues, both premalignant conditions displayed no MQ1 positivity. FIG. 5 shows strong MQ1 positivity in invasive ductal carcinoma cells and lobular carcinoma cells surrounded by MQ1 negative stroma.

Isolation of MQ-1 Clones

Screening of a cDNA expression library (from G-CCM mRNA) with the MQ1 antibody identified two clones with significant homology to the Jagged 1 protein (Sequence ID No's 1 and 2).

### Antisense Treatment Protocol

Antisense Oligonucleotide

| | |
|---|---|
| 5′-tgg gga acg cat cgc tgc-3′ | (Sequence ID No. 4) |

Antisense Control Oligonucleotide

| | |
|---|---|
| 5′-tgg gga ccg cat cgc tgc-3′ | (Sequence ID No. 5) |

The PTO linked antisense oligonucleotide was designed against the transcription initiation site and kozac sequence at the beginning of the Jaggedi gene (Accession number AF028593). The control oilgonucleotide was the same 18 mer with one base changed (therefore being the tightest control possible to generate). Both oligonucleotides were synthesized by MWG Biotech. For colony count assays G-CCM cells were seeded out into 24 well plates at 50,000 cells/well. The cells were incubated for 24 hrs in growth medium and then washed with serum free medium (SFM). The cells were then either treated with lipofectin (Invitrogen Life Technologies) alone following the standard protocol (at 5 μl/ml) or lipofectin with the antisense and antisense control oligonucleotides at a range of concentrations (0.1, 0.5 and 1.0 μM) for 16 hrs. Following treatment the cells were washed twice with SFM and then incubated in growth medium for 24 and 48 hrs. The results (FIG. 6) show that treatment with the antisense oligonucleotide at concentrations of 0.5 and 1.0 μM reduced the tumour cell population when compared to the control oligonucleotide and lipofectin alone treatment. To assess whether this was due to the induction of apoptosis similarly treated cells were harvested for their protein and examined for Parp cleavage (an indicator of apoptosis) by immunoblotting. The results (FIG. 7) clearly show a reduction in the level of Parp at 0.5 and 1.0 μM antisense oligonucleotide treatment when compared to control oligonucleotide and lipofectin alone treatment. Thus indicating that the antisense oligonucleotide treatment induces apoptosis in the G-CCM cells. To confirm this, treated G-CCM cells were also examined for the presence of cleaved Caspase 3 (another indicator of apoptosis) by immunocytochemistry. The results (FIG. 8) show that G-CCM cells treated with 1.0 μM displayed caspase 3 cleavage thus indicating that apoptosis was being induced. To demonstrate that these effects were due to the knocking out of the MQ1 proteins by the antisense oligonucleotides, treated cells were examined for the presence of the MQ1 proteins by immunocytochemistry with the MQ1 antibody. The results (FIG. 9) show that the expression levels of the MQ1 proteins is reduced by antisense oligonucleotide treatment when compared to the control oligonucleotide.

The invention described herein has potential uses as a:

Diagnostic Tool—The antibody clearly distinguishes astrocytomas from other primary brain tumours, normal cells and reactive gliosis. In addition it recognizes 60% of primary breast tumours tested.

Targeting Device—The specificity of the antibody means it can be used as a targeting device such as in radioimmunotherapy.

Therapeutic Target—The antibody itself can be used as a therapeutic agent by blocking out signaling through the MQ1/Notch pathway thus inducing apoptosis in astrocytoma cells.

The invention is not limited to the embodiments hereinbefore described which may be varied without departing from the spirit of the invention.

1. An isolated nucleic acid sequence which comprises a sequence selected from the group consisting of: Sequence ID No. 1, Sequence ID No. 2, and sequence ID No 3.

2. An isolated nucleic acid sequence according to claim 1 in which the nucleic acid sequence is a DNA sequence.

3. An isolated nucleic acid sequence according to claim 1 or 2 in which the nucleic acid sequence consists of a sequence selected from the group consisting of: Sequence ID No. 1, Sequence ID No. 2, and Sequence ID No. 3.

4. An isolated protein encoded by a nucleic acid sequence according to claim 1.

5. An isolated protein according to claim 4 in which the protein is a cell surface glycoprotein.

6. An isolated protein as claimed in claim 4 or 5 which is an oncofetal protein expressed by an astrocytoma cell.

7. An isolated protein as claimed in claim 4 having a molecular weight of approximately 200 kda.

8. An antibody which binds specifically to the protein of claim 4, and any other antibody that competes directly or by stearic hindrance therewith for said protein.

9. An antibody as claimed in claim 8 which is a monoclonal antibody.

10. An antibody as claimed in claim 8 or 9 which is a class M immunoglobulin with a kappa-light chain.

11. A fragment of the antibody of claim 8, which fragment binds specifically to a protein encoded by a nucleic acid sequence consisting of a sequence selected from the group consisting of Sequence ID No. 1, Sequence ID No. 2, and Sequence ID No. 3.

12. A method of producing an antibody to a protein comprising:
innoculating an animal with a protein according to claim 4, wherein the protein elicits an immune response in the animal to produce the antibody; and
isolating the antibody from the animal.

13. A method of producing an antibody as claimed in claim 11 in which the animal is innoculated with G-CCM cells of ECACC deposit No. 86022702.

14. A method for producing a hybridoma, comprising the step of innoculating a suitable subject with a protein according to claim 4, or an antigenic fragment thereof, and fusing cells from the subject with a myeloma cell to produce the hybridoma.

15. A method according to claim 14 in which the subject is innoculated with G-CCM cells of ECACC deposit No. 86022702.

16. A hybridoma cell obtainable according to the method of claims 14 or 15.

17. A hybridoma cell of, or derived from, ECACC Deposit No. 03073001.

18. A monoclonal antibody obtainable from a hybridoma cell of, or derived from, ECACC Deposit No. 03073001.

19. A method of detecting an astrocytoma cell in a sample of human cells, which method comprises the step of contacting the cell sample with an antibody according to claim 8 or 18, or a fragment thereof, and detecting those cells which have bound the antibody or fragment, wherein binding of the antibody or the fragment to a cell is indicative of an astrocytoma cell.

20. A method as claimed in claim 19 in which the antibody is a monoclonal antibody.

21. A method of detecting a primary breast carcinoma cell in a sample of human cells, which method comprises the step of contacting the cell sample with an antibody according to claim 8 or 18, or a fragment thereof, and detecting those cells which have bound the antibody or fragment, wherein binding of the antibody or the fragment to a cell is indicative of a primary breast carcinoma cell.

22. A method according to claim 21 in which the antibody is a monoclonal antibody.

23. A diagnostic kit for diagnosing the presence of a cell selected from the group consisting of: astrocytoma cells; malignant melanoma secondary tumour cells; and
primary breast carcinoma cells, the kit comprising a (primary) antibody according to claim 8 or 18, or a fragment thereof.

24. A diagnostic kit as claimed in claim 23 in which the antibody comprises a detectable label.

25. A diagnostic kit as claimed in claim 23 in which the kit comprises a secondary antibody which specifically binds the (primary) antibody, which secondary antibody comprises a detectable label.

26. A biological targeting device comprising an antibody according to claim 8 or 18, or a fragment thereof, and a therapeutic ligand.

27. A therapeutic antibody comprising an antibody according to claim 8 or 18, or a fragment thereof.

28. A method of treating cancer in an individual by inducing apoptosis in cells in the individual which express an MQ1 protein, which method comprises a step of treating an individual with an antibody of claim 8 or 18, or a fragment thereof.

29. A method according to claim 28 in which the cancer is selected from the group consisting of: malignant astrocytomas; malignant melanoma secondary tumours; and primary breast carcinomas.

30. A method according to claim 28 in which the antibody is a monoclonal antibody.

31. A method as claimed in claim 28 in which the antibody is humanised.

32. A polynucleotide which is anti-sense to an isolated nucleic acid sequence of claim 1.

33. An anti-sense polynucleotide as claimed in claim 32 comprising the sequence of Sequence ID No. 4.

34. An anti-sense polynucleotide as claimed in claim 32 consisting of the sequence of Sequence ID No. 4.

35. Method of treating cancer in an individual by inducing apoptosis in cells in the individual which express an MQ1 protein, which method comprises a step of treating an individual with an anti-sense polynucleotide of any of claims 32 to 34.

36. A method according to claim 35 in which the cancer is selected from the group consisting of: malignant astrocytomas; malignant melanoma secondary tumours; and primary breast carcinomas.

37. An isolated protein encoded by a nucleic acid sequence according to claim 2.

38. An isolated protein encoded by a nucleic acid sequence according to claim 3.
